(19) United States
(12) Patent Application Publication
(10) Pub. No.: US 2017/0107544 A1
(43) Pub. Date: Apr. 20, 2017

(54) MICROORGANISM CAPABLE OF PRODUCING 1,4-BUTANEDIOL AND METHOD OF PRODUCING 1,4-BUTANEDIOL USING THE SAME

(71) Applicant: Samsung Electronics Co., Ltd., Suwon-si (KR)

(72) Inventors: Yukyung Jung, Hwaseong-si (KR); Kwangmyung Cho, Seongnam-si (KR); Jinhwan Park, Suwon-si (KR); Hwayoung Cho, Hwaseong-si (KR)

(21) Appl. No.: 15/384,166

(22) Filed: Dec. 19, 2016

Related U.S. Application Data
(62) Division of application No. 14/500,269, filed on Sep. 29, 2014.

(30) Foreign Application Priority Data
Sep. 27, 2013 (KR) 10-2013-0115576

(51) Int. Cl. C12P 7/18; C12N 15/70; C12N 9/04

(52) U.S. Cl. CPC C12P 7/18; C12N 9/0006; C12Y 101/01001; C12N 15/70

(57) ABSTRACT

A microorganism capable of producing 1,4-butanediol and a method of producing 1,4-butanediol using the same.

### RELATED APPLICATIONS

This application claims the benefit of Korean Patent Application No. 10-2013-0115576, filed on Sep. 27, 2013, the entire disclosure of which is hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: 72,258 ASCII (Text) file named “718196_ST25.TXT” created Sep. 23, 2014.

### BACKGROUND

1. Field

The present disclosure relates to a microorganism capable of producing 1,4-butanediol and a method of producing 1,4-butanediol using the same.

2. Description of the Related Art

1,4-butanediol (1,4-BDO) may be used as an industrial solvent or for the preparation of several types of plastics, elastic fiber, and polyurethane. In organic chemistry, 1,4-BDO may be used for synthesis of γ(gamma)-butyrolactone. In the presence of phosphoric acid and at a high temperature, 1,4-BDO may be dehydrated to form tetrahydrofuran, an important solvent.

Currently, 1,4-BDO is produced from acetylene, maleic anhydride, and propylene oxy, which are precursors of petrochemistry. However, as oil price increases, an alternative method of producing 1,4-BDO is required.

Therefore, a method of efficiently preparing 1,4-BDO by using a microorganism is required.

### SUMMARY

Provided is a genetically engineered microorganism of which capability of producing 1,4-BDO is improved. The genetically engineered microorganism comprises a genetic modification that decreases activity of converting pyruvate to lactate, activity of converting acetyl-CoA to ethanol, activity of converting oxaloacetate to malate, or a combination thereof in the genetically engineered microorganism in comparison to the activity in a parent microorganism not having the genetic modification; and the genetically engineered microorganism comprises a genetic modification that increases activity of converting succinate to 4-hydroxybutyrate (4HB) and activity of converting 4HB to 1,4-butanediol (1,4-BDO) in the genetically engineered microorganism in comparison to the activity in the parent microorganism not having the genetic modification.

Also provided is a method of efficiently producing 1,4-BDO using the microorganism. The method comprises culturing in the presence of succinate a microorganism having the genetic modification that increases activity of converting succinate to 4-HB and activity of converting 4HB to 1,4-BDO, wherein the microorganism produces 1,4-BDO; and recovering 1,4-BDO from the culture.

### BRIEF DESCRIPTION OF THE DRAWINGS

These and/or other aspects will become apparent and more readily appreciated from the following description of the embodiments, taken in conjunction with the accompanying drawings in which:

FIG. 1 is a diagram showing the 1,4-BDO production pathway of one embodiment of the microorganism;

FIG. 2 is a diagram showing the pMloxC vector map;

FIG. 3 is a diagram showing the pTac15k sucCD-sucD-4hbd vector map; and

FIG. 4 is a diagram showing the pTrc99a ald-cat2 vector map.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items.

An aspect of the present disclosure provides a genetically engineered microorganism comprises a genetic modification that causes a decrease in the activity of converting pyruvate to lactate, activity of converting acetyl-CoA to ethanol, activity of converting oxaloacetate to malate, or a combination thereof in comparison to the activity in the parent microorganism not having the genetic modification; and the genetically engineered microorganism comprises a genetic modification that causes an increase in the activity of converting succinate to 4-hydroxybutyrate (4HB) and activity of converting 4HB to 1,4-butanediol (1,4-BDO) that is increased in comparison to the activity in the parent microorganism not having the genetic modification. The parent microorganism is the microorganism that is to be genetically modified to provide the genetically engineered microorganism.

The microorganism may comprise a genetic modification that decreases activity of converting pyruvate to lactate, activity of converting acetyl-CoA to ethanol, activity of converting oxaloacetate to malate, or a combination thereof, and a genetic modification that increases activity of converting succinate to 4-hydroxybutyrate (4HB) and activity of converting 4HB to 1,4-butanediol (1,4-BDO).

The microorganisms may contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

The genetic modification may include mutation of the gene or a regulatory region of the gene (e.g., operator, promoter or terminator regions of the gene), or a part thereof, sufficient to disrupt gene function or the expression of a functional gene product. Mutations include substitutions, additions, and deletions of one or more bases or one or more nucleotide in the gene or its regulator regions. As a result, the gene is not expressed or has a reduced amount of expression, or the activity of the encoded protein or enzyme is reduced or eliminated. The disruption of the gene may be accomplished by any suitable genetic engineering technique, such as homologous recombination, mutation induction, or molecular evolution. When a cell includes a plurality of copies of the same gene or at least two different polypeptide paralogs, at least one gene may be disrupted. The genetic modification may also include an introduction of a gene into a host cell.

Decrease of activity herein may refer to decrease of activity of a mentioned protein included in a microorganism. The decrease of activity may include not only decrease of expression of one or more genes encoding a mentioned protein but also decrease in activity due to other causes, such as decrease of specific activity of a protein itself. The decrease of activity may refer to decrease which is caused by inactivation or attenuation of a gene encoding a mentioned protein. The “decrease” may refer to a relative decrease of activity in comparison with that of a parent microorganism not having the genetic modification (e.g., not having the given genetic modification).

The term “inactivation” herein may mean that a gene is not expressed or a gene is expressed but a product of the expressed gene is not active. The term “attenuation” may mean that expression of a gene is decreased to a level lower than an expression level of a strain that does not have the genetic modification, or that a gene is expressed but a product of the expressed gene has a decreased activity. The inactivation or the attenuation may be caused by homologous recombination. The inactivation or attenuation may be performed by transforming a vector including a part of a sequence of the gene into a cell, culturing the cell so that homologous recombination of the sequence may occur with an endogenous gene of the cell, and then selecting a cell in which homologous recombination has occurred using a selection marker.

The microorganism may be a microorganism which belongs to Escherichia genus, or Corynebacterium genus. The microorganism belonging to Escherichia genus may be E. coli. The microorganism may be capable of producing 1,4-BDO. In the microorganism, a gene encoding lactate dehydrogenase (LDH) converting pyruvate to lactate, for example, lactate dehydrogenase A (LDHA), a gene encoding alcohol dehydrogenase (ADH) converting acetyl-CoA to ethanol, for example, alcohol dehydrogenase 1 (ADH1), a gene encoding malate dehydrogenase converting oxaloacetate to malate, or a combination thereof may be inactivated or attenuated to an extent sufficient to produce 1,4-BDO. Activity of the enzymes mentioned above may be decreased, with respect to that of an appropriate control group species, for example, a microorganism that does not have the genetic modification, by about 75% or more, by about 80% or more, by about 85% or more, by about 90% or more, by about 95% or more, or by about 100%. In the microorganism, the microorganism may include a genetic modification that increases activity of converting succinate to 4HB and/or activity of converting 4HB to 1,4-BDO to an extent sufficient to produce 1,4-BDO. In the microorganism, activity of converting succinate to 4HB and/or activity of converting 4HB to 1,4-BDO may be increased to an extent sufficient to produce 1,4-BDO. The activity may be increased, with respect to that of a control group, by about 100% or more, by about 110% or more, by about 120% or more, by about 130% or more, by about 140% or more, by about 150% or more, by about 160% or more, by about 170% or more, by about 200% or more, by about 300% or more, by about 500% or more, by about 1000% or more, by about 2000% or more, or by about 10,000% or more.

A polypeptide converting pyruvate to lactate, for example, LDH, may be an enzyme catalyzing a reaction of reversibly converting pyruvate to lactate by using a reduction of NAD(P)⁺ to NAD(P)H. The LDH may be an enzyme classified as EC.1.1.1.27 or EC 1.1.2.3. The LDH may have an amino acid sequence of SEQ ID NO:1. The gene encoding the LDH may have a nucleotide sequence of SEQ ID NO:2. The gene may be E. coli ldhA encoding NADH-linked LDH.

A polypeptide converting acetyl-CoA to ethanol may be ADH. The ADH may be an enzyme reversibly converting acetyl-CoA to ethanol along with an oxidation of NADH to NAD⁺. The ADH may be an enzyme classified as EC 1.1.1.1. The ADH may have an amino acid sequence of SEQ ID NO:3. The gene encoding the ADH may have a nucleotide sequence of SEQ ID NO:4. The gene may be E. coli adhA encoding NADH-linked ADH.

The malate dehydrogenase may be an enzyme reversibly converting oxaloacetate to malate by using a reduction of NAD(P)⁺ to NAD(P)H. The malate dehydrogenase may be an enzyme classified as EC 1.1.1.37. The malate dehydrogenase may have an amino acid sequence of SEQ ID NO:5. The gene encoding the malate dehydrogenase may have a nucleotide sequence of SEQ ID NO:6. The gene may be E. coli mdh encoding NADH-linked malate dehydrogenase.

In the microorganism, activity of converting succinate to 4HB may be increased by an increased expression of a gene encoding a polypeptide converting succinate to succinyl-CoA, a gene encoding a polypeptide converting succinyl-CoA to succinic semialdehyde (SSA), a gene encoding a polypeptide converting SSA to 4HB, or a combination thereof.

The increased expression may be caused by an increased expression of one or more endogenous genes or by an introduction of one or more exogenous genes (e.g., heterologous genes). The increased expression of an endogenous gene may be caused by amplification of the gene or by mutation of a regulatory region. The exogenous gene may be a homologous or a heterologous gene.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

A polypeptide converting succinate to succinyl-CoA may be an enzyme reversibly converting succinate to succinyl-CoA. The enzyme may catalyze a reaction which is represented by the reaction formula: succinate+CoA+NTP⇄succinyl-CoA+Pi+NDP. The NTP may be an ATP or a GTP. The enzyme may be succinyl-CoA synthetase (also referred to as succinyl-CoA ligase or succinate thiokinase). The enzyme may be succinyl-CoA synthetase (e.g., SucCD) or succinyl-CoA:coenzyme A transferase (e.g., Cat1). The succinyl-CoA synthetase may be an enzyme classified as EC 6.2.1 (acid-thiol ligase), for example, EC 6.2.1.4 or EC 6.2.1.5. The SucCD may have an amino acid sequence of SEQ ID NO:7.

A polynucleotide encoding the succinyl-CoA synthetase or the SucCD may have a nucleotide sequence of SEQ ID NO:8. The Cat1 may be an enzyme classified as EC. 2.8.3 (CoA-transferase), for example, EC.2.8.3.18 (succinyl-CoA:acetate CoA-transferase). The Cat1 may be an enzyme reversibly converting succinate and acetyl-CoA to succinyl-CoA and acetate. A gene encoding the SucCD may be derived from E. coli. The Cat1 may have an amino acid sequence of SEQ ID NO:9. A polynucleotide encoding the Cat1 may have a nucleotide sequence of SEQ ID NO:10. A polynucleotide encoding the SucCD may be derived from E. coli. A gene encoding the Cat1 may be derived from Clostridium kluyveri./

A polynucleotide converting succinyl-CoA to succinic semialdehyde may be CoA-dependent succinate semialdehyde dehydrogenase (e.g., SucD). The SucD may be oxidoreductase (EC.1.2.1.76) which uses NAD or NADP as an electron accepter, for example, an enzyme converting acyl-CoA to aldehyde. The SucD may have an amino acid sequence of SEQ ID NO:11. A polynucleotide encoding the SucD may have a nucleotide sequence of SEQ ID NO:12. The SucD and a gene encoding the SucD may be derived from Porphyromonas gingivalis.

A polypeptide converting SSA to 4HB may be 4-hydroxybutyrate dehydrogenase (4Hbd). The 4Hbd may be oxidoreductase (EC.1.1.1) which uses NAD or NADP as an electron acceptor, for example, an enzyme converting ketone to hydroxyl and/or converting aldehyde to alcohol. The 4Hbd may have an amino acid sequence of SEQ ID NO:13. A polynucleotide encoding the 4Hbd may have a nucleotide sequence of SEQ ID NO:14. The 4Hbd and a gene encoding the 4Hbd may be derived from Porphyromonas gingivalis.

In the microorganism, activity of converting succinate to 4HB may be increased by an introduction of a gene encoding a polypeptide converting succinate to succinyl-CoA, for example, sucCD, a gene encoding a polypeptide converting succinyl-CoA to SSA, for example, sucD, a gene encoding a polypeptide converting SSA to 4HB, for example, 4hbd, or a combination thereof. Thus, the microorganism may include an exogenous gene encoding a polypeptide converting succinate to succinyl-CoA, for example, sucCD, an exogenous encoding a polypeptide converting succinyl-CoA to SSA, for example, sucD, an exogenous encoding a polypeptide converting SSA to 4HB, for example, 4hbd, or a combination thereof.

In the microorganism, the microorganism may have a genetic modification that increases expression of a gene encoding a polypeptide converting 4HB to 4-hydroxybutyryl-CoA (4HB-CoA), a gene encoding a polypeptide converting 4HB-CoA to 1,4-BDO, or a combination thereof. In the microorganism, activity of converting 4HB to 1,4-BDO may be increased by an increased expression of a gene encoding a polypeptide converting 4HB to 4-hydroxybutyryl-CoA (4HB-CoA), a gene encoding a polypeptide converting 4HB-CoA to 1,4-BDO, or a combination thereof. The increased expression may be caused by an increased expression of one or more endogenous genes or by an introduction of one or more exogenous genes. The increased expression of an endogenous gene may be caused by amplification of the gene or by mutation of a regulatory region. The exogenous gene may be a homologous or a heterologous gene.

A polypeptide converting 4HB to 4HB-CoA may be 4-hydroxybutyryl-CoA:acetyl-CoA transferase (Cat2). The Cat2 may be an enzyme classified as a CoA-transferase (EC.2.8.3). The Cat2 may have an amino acid sequence of SEQ ID NO:15. A polynucleotide encoding the Cat2 may have a nucleotide sequence of SEQ ID NO:16. The Cat2 and a gene encoding the Cat2 may be derived from Porphyromonas gingivalis.

A polypeptide converting 4HB-CoA to 1,4-BDO may be alcohol dehydrogenase (e.g., AdhE2) and/or aldehyde dehydrogenase (Ald). The Adhe2 and Ald may convert 4HB-CoA to 4-hydroxyburyl aldehyde and then to 1,4-BDO. The AdhE2 and Ald may an enzyme converting acyl-CoA to alcohol in two steps (EC.1.1.1) or an enzyme classified as EC.1.2.1.3. The AdhE2 may have an amino acid sequence of SEQ ID NO:17. A polynucleotide encoding the AdhE2 may have a nucleotide sequence of SEQ ID NO:18. The AdhE2 and a gene encoding the AdhE2 may be derived from Clostridium acetobutylicum. The Ald may have an amino acid sequence of SEQ ID NO:19. A polynucleotide encoding the Ald may have a nucleotide sequence of SEQ ID NO:20. The Ald and a gene encoding the Ald may be derived from Clostridium beijerinckii.

The microorganism may include a genetic modification that increases activity of converting 4HB to 1,4-BDO. The microorganism may include one or more exogenous genes encoding a polypeptide converting 4HB to 4HB-CoA, for example, cat2, exogenous genes encoding a polypeptide converting 4HB-CoA to 1,4-BDO, for example, ald, or a combination thereof. Activity of converting 4HB to 1,4-BDO may be increased by an introduction of one or more genes encoding a polypeptide converting 4HB to 4HB-CoA, for example, cat2, a gene encoding a polypeptide converting 4HB-CoA to 1,4-BDO, for example, ald, or a combination thereof. The microorganism may not include an exogenous gene encoding α-ketoglutarate decarboxylase (sucA).

The microorganism may be an E. coli in which a gene encoding LDH converting pyruvate to lactate, a gene encoding ADH converting acetyl-CoA to ethanol, a gene encoding malate dehydrogenase converting oxaloacetate to malate, or a combination thereof is inactivated or attenuated, and include a genetic modification that increases activity of converting succinate to 4-HB and activity of converting 4HB to 1,4-BDOand the activity of converting 4HB to 1,4-BDO. For example, the microorganism may include one or more exogenous genes encoding a polypeptide converting succinate to succinyl-CoA, one or more exogenous genes encoding a polypeptide converting succinyl-CoA to SSA, one or more exogenous genes encoding a polypeptide converting SSA to 4HB, or a combination thereof and include one or more exogenous genes encoding a polypeptide converting 4HB to 4HB-CoA, one or more exogenous genes encoding a polypeptide converting 4HB-CoA to 1,4-BDO, or a combination thereof.

Another aspect of the present disclosure provides a method of producing 1,4-BDO including culturing in the presence of succinate a microorganism having a genetic modification that increases activity of converting succinate to 4-HB and activity of converting 4HB to 1,4-BDO; and recovering 1,4-BDO from the culture.

The method includes culturing in the presence of succinate a microorganism having a genetic modification that increases activity of converting succinate to 4-HB and activity of converting 4HB to 1,4-BDO.

The microorganism may be a microorganism which belongs to Escherichia genus, or Corynebacterium genus. The microorganism belonging to Escherichia genus may be E. coli. Corynebacterium genus may be Corynebacterium glutamicum. The microorganism may be capable of producing 1,4-BDO. In the microorganism, one or more genes encoding LDH converting pyruvate to lactate, for example, LDHA, one or more genes encoding ADH converting acetyl-CoA to ethanol, for example, ADH1, one or more genes encoding malate dehydrogenase converting oxaloacetate to malate, or a combination thereof may be inactivated or attenuated to an extent sufficient to produce 1,4-BDO. Activity of the enzymes mentioned above may be decreased, with respect to that of an appropriate control group species, for example, a microorganism which is not engineered, by about 75% or more, by about 80% or more, by about 85% or more, by about 90% or more, by about 95% or more, or by about 100%. In the microorganism, activity of converting succinate to 4HB and/or activity of converting 4HB to 1,4-BDO may be increased to an extent sufficient to produce 1,4-BDO. The activity may be increased, with respect to that of a control group, by about 110% or more, by about 110% or more, by about 120% or more, by about 130% or more, by about 140% or more, by about 150% or more, by about 160% or more, by about 170% or more, by about 200% or more, by about 300% or more, by about 500% or more, by about 1000% or more, by about 2000% or more, or by about 10,000% or more.

A polypeptide converting pyruvate to lactate, for example, LDH, may be an enzyme catalyzing a reaction of reversibly converting pyruvate to lactate by using a reduction of NAD(P)⁺ to NAD(P)H. The LDH may be an enzyme classified as EC.1.1.1.27 or EC 1.1.2.3. The LDH may have an amino acid sequence of SEQ ID NO:1. The gene encoding the LDH may have a nucleotide sequence of SEQ ID NO:2. The gene may be E. coli ldhA encoding NADH-linked LDH.

A polypeptide converting acetyl-CoA to ethanol may be ADH. The ADH may be an enzyme reversibly converting acetyl-CoA to ethanol along with an oxidation of NADH to NAD⁺. The ADH may be an enzyme classified as EC 1.1.1.1. The ADH may have an amino acid sequence of SEQ ID NO:3. The gene encoding the ADH may have a nucleotide sequence of SEQ ID NO:4. The gene may be E. coli adhA encoding NADH-linked ADH.

The malate dehydrogenase may be an enzyme reversibly converting oxaloacetate to malate by using a reduction of NAD(P)⁺ to NAD(P)H. The malate dehydrogenase may be an enzyme classified as EC 1.1.1.37. The malate dehydrogenase may have an amino acid sequence of SEQ ID NO:5. The gene encoding the malate dehydrogenase may have a nucleotide sequence of SEQ ID NO:6. The gene may be E. coli mdh encoding NADH-linked malate dehydrogenase.

In the microorganism, activity of converting succinate to 4HB may be increased by an increased expression of a gene encoding a polypeptide converting succinate to succinyl-CoA, a gene encoding a polypeptide converting succinyl-CoA to SSA, a gene encoding a polypeptide converting SSA to 4HB, or a combination thereof.

The increased expression may be caused by an increased expression of one or more endogenous genes or by an introduction of one or more foreign genes. The increased expression of an endogenous gene may be caused by amplification of the gene or by mutation of a regulatory region. The foreign gene may be an endogenous or an exogenous gene.

A polypeptide converting succinate to succinyl-CoA may be an enzyme reversibly converting succinate to succinyl-CoA. The enzyme may catalyze a reaction which is represented by the reaction formula: succinate+CoA+NTP⇄succinyl-CoA+Pi+NDP. The NTP may be an ATP or a GTP. The enzyme may be succinyl-CoA synthetase (also referred to as succinyl-CoA ligase or succinate thiokinase). The enzyme may be SucCD or Cat1. The succinyl-CoA synthetase may be an enzyme classified as EC 6.2.1 (acid-thiol ligase), for example, EC 6.2.1.4 or EC 6.2.1.5. The SucCD may have an amino acid sequence of SEQ ID NO:7.

A polynucleotide encoding the succinyl-CoA synthetase or the SucCD may have a nucleotide sequence of SEQ ID NO:8. The Cat1 may be an enzyme classified as EC. 2.8.3 (CoA-transferase), for example, EC.2.8.3.18. The Cat1 may be an enzyme reversibly converting succinate+acetyl-CoA to succinyl-CoA+acetate. A gene encoding the SucCD may be derived from E. coli. The Cat1 may have an amino acid sequence of SEQ ID NO:9. A polynucleotide encoding the Cat1 may have a nucleotide sequence of SEQ ID NO:10. A polynucleotide encoding the SucCD may be derived from E. coli. A gene encoding the Cat1 may be derived from Clostridium kluyveri.

A polynucleotide converting succinyl-CoA to succinic semialdehyde may be SucD. The SucD may be oxidoreductase (EC.1.2.1) which uses NAD or NADP as an electron accepter, for example, an enzyme converting acyl-CoA to aldehyde. The SucD may have an amino acid sequence of SEQ ID NO:11. A polynucleotide encoding the SucD may have a nucleotide sequence of SEQ ID NO:12. The SucD and a gene encoding the SucD may be derived from Porphyromonas gingivalis.

A polypeptide converting SSA to 4HB may be 4HBd. The 4HBd may be oxidoreductase (EC.1.2.1) which uses NAD or NADP as an electron accepter, for example, an enzyme converting ketone to hydroxyl or converting aldehyde to alcohol. The 4HBd may have an amino acid sequence of SEQ ID NO:13. A polynucleotide encoding the 4HBd may have a nucleotide sequence of SEQ ID NO:14. The 4HBd and a gene encoding the 4HBd may be derived from Porphyromonas gingivalis.

In the microorganism, activity of converting succinate to 4HB may be increased by an introduction of one or more genes encoding a polypeptide converting succinate to succinyl-CoA, for example, sucCD, one or more genes encoding a polypeptide converting succinyl-CoA to SSA, for example, sucD, one or more genes encoding a polypeptide converting SSA to 4HB, for example, 4hbd, or a combination thereof.

In the microorganism, activity of converting 4HB to 1,4-BDO may be caused by an increased expression of a polypeptide converting 4HB to 4HB-CoA, a polypeptide converting 4HB-CoA to 1,4-BDO, or a combination thereof. The increased expression may caused by an increased expression of an endogenous gene or by an introduction of a foreign gene. The increased expression of an endogenous gene may be caused by amplification of the gene or by mutation of a regulatory region. The foreign gene may be an endogenous or an exogenous gene.

A polypeptide converting 4HB to 4HB-CoA may be Cat2. The Cat2 may be an enzyme classified as a CoA-transferase (EC.2.8.3). The Cat2 may have an amino acid sequence of SEQ ID NO:15. A polynucleotide encoding the Cat2 may have a nucleotide sequence of SEQ ID NO:16. The Cat2 and a gene encoding the Cat2 may be derived from Porphyromonas gingivalis.

A polypeptide converting 4HB-CoA to 1,4-BDO may be AdhE2 and/or Ald. The Adhe2 and Ald may convert 4HB-CoA to 4-hydroxyburyl aldehyde and then to 1,4-BDO. The Adhe2 and Ald may an enzyme converting acyl-CoA to alcohol in two steps (EC.1.1.1) or an enzyme classified as EC.1.2.1.3. The AdhE2 may have an amino acid sequence of SEQ ID NO:17. A polynucleotide encoding the AdhE2 may have a nucleotide sequence of SEQ ID NO:18. The AdhE2 and a gene encoding the AdhE2 may be derived from Clostridium acetobutylicum. The Ald may have an amino acid sequence of SEQ ID NO:19. A polynucleotide encoding the Ald may have a nucleotide sequence of SEQ ID NO:20. The Ald and a gene encoding the Ald may be derived from Clostridium beijerinckii.

Activity of converting 4HB to 1,4-BDO may be increased by an introduction of one or more genes encoding a polypeptide converting 4HB to 4HB-CoA, for example, cat2, one or more genes encoding a polypeptide converting 4HB-CoA to 1,4-BDO, for example, ald, or a combination thereof. The microorganism may not include an exogenous gene encoding sucA.

The microorganism may be an E. coli in which one or more genes encoding LDH converting pyruvate to lactate, one or more genes encoding ADH converting acetyl-CoA to ethanol, one or more genes encoding malate dehydrogenase converting oxaloacetate to malate, or a combination thereof is inactivated or attenuated, and activity of converting succinate to 4-HB and activity of converting 4HB to 1,4-BDO are increased, wherein the activity of converting succinate to 4-HB is increased by an introduction of one or more genes encoding a polypeptide converting succinate to succinyl-CoA, one or more genes encoding a polypeptide converting succinyl-CoA to SSA, one or more genes encoding a polypeptide converting SSA to 4HB, or a combination thereof and the activity of converting 4HB to 1,4-BDO is increased by an introduction of one or more genes encoding a polypeptide converting 4HB to 4HB-CoA, one or more genes encoding a polypeptide converting 4HB-CoA to 1,4-BDO, or a combination thereof.

The culturing may be performed with an appropriate culture medium and under culture conditions known in this art. The culture medium and culture conditions may be adjusted according to the selected microorganism. The culturing method may include batch culturing, continuous culturing, fed-batch culturing or a combination thereof. Succinate may be fed during the culturing. The fed succinate may be succinic acid or a salt thereof. The salt may be a sodium salt. The succinate may be fed at the beginning of the culturing, or one or two or more times after beginning the culturing, for example, a few times. The succinate feeding concentration may be adjusted depending on cell concentration, feeding time, or other considerations. The feeding concentration may be, for example, from about 0.1 g/L to about hundreds g/L, for example, from about 0.1 g/L to about 500 g/L, from about 0.1 g/L to about 400 g/L, from about 0.1 g/L to about 300 g/L, from about 0.1 g/L to about 200 g/L, from about 0.1 g/L to about 100 g/L, from about 0.1 g/L to about 50 g/L, from about 1.0 g/L to about 500 g/L, from about 5.0 g/L to about 400 g/L, from about 10 g/L to about 300 g/L, from about 50 g/L to about 200 g/L, from about 1 g/L to about 100 g/L, or from about 5 g/L to about 50 g/L. For example, the succinate may be fed when one or more genes producing 4-HB or 1,4-BDO from succinate is sufficiently expressed.

The culture medium may include various carbon sources, nitrogen sources, and trace elements. The carbon source may include a carbohydrate such as glucose, sucrose, lactose, fructose, maltose, starch, and cellulose, a lipid such as soybean oil, sunflower oil, castor oil, and coconut oil, a fatty acid such as palmitic acid, stearic acid, and linoleic acid, an organic acid such as acetic acid or a combination thereof. The culturing may be performed by using glucose as a carbon source. The nitrogen source may include an organic nitrogen source such as peptone, yeast extract, meat extract, malt extract, corn steep liquid, and soybean, an inorganic nitrogen source such as urea, ammonium sulfate, ammonium chloride, ammonium phosphate, ammonium carbonate, and ammonium nitrate or a combination thereof. The culture medium may include as a phosphorous source, for example, potassium dihydrogen phosphate, dipotassium phosphate, a sodium-containing salt corresponding to potassium dihydrogen phosphate, and dipotassium phosphate, and a metal salt such as magnesium sulfate and iron sulfate. The culture medium or an individual component may be added to the culturing solution in a batch mode or a continuous mode.

In addition, a compound such as ammonium hydroxide, potassium hydroxide, ammonia, phosphoric acid or sulfuric acid may be added to the microorganism culturing solution in an appropriate mode to adjust pH of the culture solution. In addition, a defoaming agent such as fatty acid polyglycol ester may be used during the culturing to repress bubble formation.

The culturing may be performed under aerobic, microaerobic, or anaerobic conditions. The term “aerobic condition” refers to culturing conditions under which a culture medium may exchange oxygen-containing air. In addition, the term “aerobic condition” may include culturing in a culture medium having a dissolved oxygen concentration which is, for example, about 1% or higher, about 10% or higher, about 30% or higher, about 40% or higher, about 50% or higher, about 60% or higher, about 70% or higher, about 80% or higher, about 90% or higher, or about 100% of a saturated concentration. The culturing may include culturing in a culture medium having a dissolved oxygen concentration which is from about 1% to about 100%, from about 1% to about 50%, from about 50% to about 100%, from about 60% to about 100%, from about 70% to about 100%, from about 80% to about 100%, from about 90% to about 100%, from about 95% to about 100%, or about 100% of a saturated concentration. The aerobic conditions may be maintained throughout the culturing. The saturated concentration may be a saturated concentration at a temperature at which the culturing is performed, for example, about 30° C. For an efficient converting of externally fed succinate to 1,4-BDO, the culturing conditions may be aerobic.

In the culturing, the temperature of the culturing solution may be from about 20° C. to about 45° C., for example, from about 22° C. to about 42° C. or from about 25° C. to about 40° C. The culturing may be continued until a desired production amount of 1,4-BDO is acquired.

Hereinafter, the present disclosure will be described in further detail with reference to examples. It will be obvious to a person having ordinary skill in the art that these examples are illustrative purposes only and are not to be construed to limit the scope of the present disclosure.

### Example 1: Preparation of Microorganism Capable of Efficiently Producing 1,4-BDO

In Example 1, a microorganism capable of producing 1,4-BDO in which an LDH gene, a ADH gene, and a malate dehydrogenase gene were inactivated was prepared.

As described below, in an E. coli W (ATCC 9637), ldhA which is the gene encoding an enzyme involved in a production pathway of lactate (SEQ ID NOS:1 and 2), which is a major side product under anaerobic conditions, adhE which is the gene encoding an enzyme involved in a production pathway of ethanol (SEQ ID NOS:3 and 4), and mdh which is the gene encoding an enzyme involved in a production pathway of succinate (SEQ ID NOS:5 and 6) were deleted. Deletion of the mdh gene was performed to show that 1,4-BDO may be produced by not utilizing endogenous succinate but rather relying solely on externally fed succinate as a substrate. To the E. coli W which was mutated as described herein, a recombinant vector including a gene encoding a polypeptide having activity of converting succinate to 4-HB and a gene encoding a polypeptide having activity of converting 4-HB to 1,4-BDO was transformed to prepare a mutant E. coli W capable of efficiently producing 1,4-BDO.

1.1 Preparation of Microorganism in which ldhA, adhE, and mdh Genes are Deleted to Block Production of Side Products (Lactate, and Ethanol) and Succinate

In an E. coli W (ATCC 9637), ldhA, adhE, and mdh genes were deleted by a first-step gene inactivation method (Warner et al., PNAS, 6:97(12):6640-6645 (2000)) by using primers described below and the obtained strain was named as WΔmdhΔldhΔadh E.

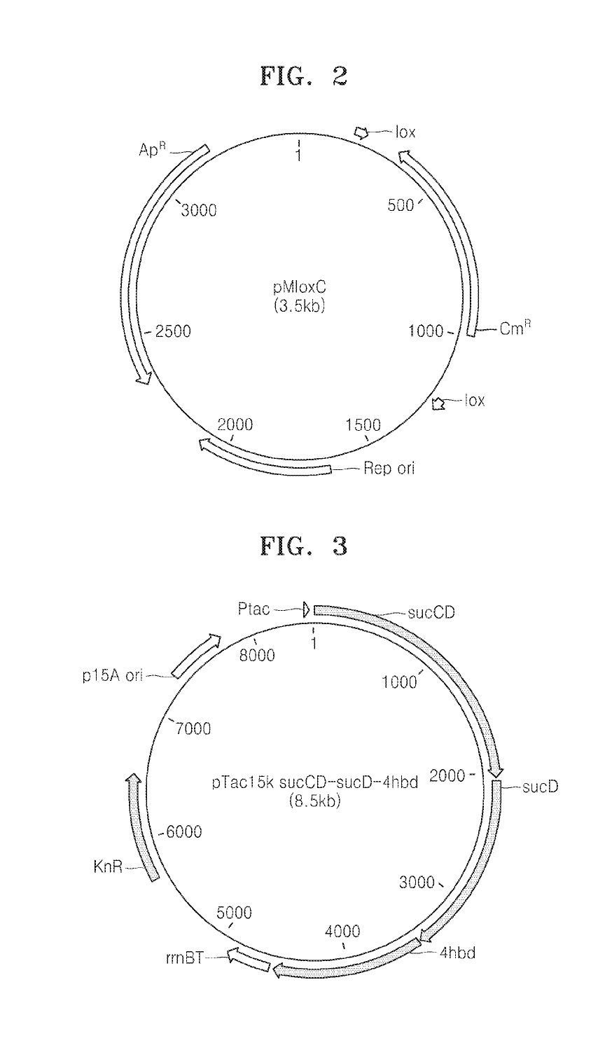

To delete the ldhA gene, a polymerase chain reaction (PCR) was performed by using a pMloxC vector (Lee, K. H. et al., Molecular systems biology 3, 149 (2007)) (FIG. 2) as a template and primers having sequences of SEQ ID NOS:21 and 22. The obtained DNA fragment was introduced by electroporation to an electroporation-competent cell of a W strain in which A-red recombinase was expressed to obtain an ldhA gene-deleted mutant strain. To verify deletion of the ldhA gene, a colony PCR was performed by using primers having sequences of SEQ ID NOS:23 and 24.

In addition, primers having sequences of SEQ ID NOS: 25 and 26 were sequentially used to delete the adhE gene by the same method and primers having sequences of SEQ ID NOS: 27 and 28 were used to verify deletion of the adhE gene.

In addition, primers having sequences of SEQ ID NOS: 29 and 30 were sequentially used to delete the mdh gene by the same method and primers having sequences of SEQ ID NOS: 31 and 32 were used to verify deletion of the mdh gene.

1.2 Introduction of sucCD, sucD, 4hbd, ald, and cat2 Genes

To the E. coli W mutant strain (WΔmdhΔldhΔadhE) obtained in Example 1.1 above, sucCD (SEQ ID NOS:7 and 8), sucD (SEQ ID NOS:11 and 12), 4hbd (SEQ ID NOS:13 and 14), cat2 (SEQ ID NOS:15 and 16), and ald (SEQ ID NOS:17 and 18) genes were introduced. The sucCD gene was derived from an E. coli, the sucD, 4hbd, and cat2 genes were derived from Porphyromonas gingivalis, and the ald gene was derived from Clostridium beijerinckii.

A PCR was performed by using a genome DNA of E. coli MG1655 as a template and by using primers having sequences of SEQ ID NOS: 33 and 34 to obtain the sucCD gene. Next, the Porphyromonas gingivalis-derived sucD, 4hbd, and cat2 genes and Clostridium beijerinckii-derived ald gene were prepared by gene synthesis (Cosmogenetech co, Ltd., Korea).

The obtained sucCD gene was introduced to pTac15k (Qian, Z. -G. et al., Biotechnol. Bioeng. 104(4):651-662 (2009)) by using restriction enzymes which were EcoRI and KpnI to prepare a pTac15k sucCD vector. The pTac15k sucCD vector was cleaved by using the restriction enzyme KpnI and the resulting fragment was used as a vector DNA fragment. In addition, both a sucD gene fragment obtained by performing a PCR using primers having sequences of SEQ ID NOS:35 and 36 and a 4hbd gene fragment obtained by performing a PCR using primers having sequences of SEQ ID NOS:37 and 38 were used as templates to perform a PCR using sequences of SEQ ID NOS:35 and 38. The obtained DNA fragment including the sucD and 4hbd genes was used as an insertion DNA fragment and linked with the pTac15k sucCD vector by using InFusion Cloning Kit (Clontech Laboratories, Inc., USA) to prepare a pTac15ksucCD-sucD-4hbd vector (FIG. 3).

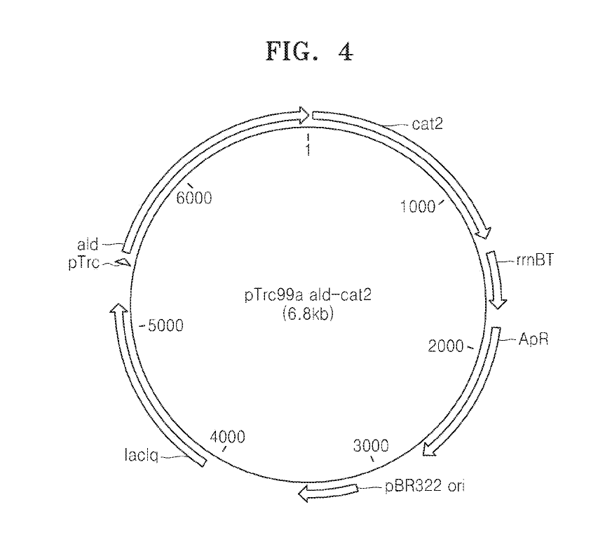

In addition, the obtained ald gene was introduced to pTrc99a (AP Biotech) by using restriction enzymes which were NcoI and EcoRI to prepare a pTrc99a ald vector. The pTrc99a ald vector was cleaved by using the restriction enzymes, which were EcoRI and HindIII, and then the cat2 gene was introduced to prepare a pTrc99a ald-cat2 (FIG. 4).

To the E. coli W mutant strain ΔmdhΔldhΔadhE, the pTac15k sucCD-sucD-4hbd vector and the pTrc99a ald-cat2 vector were introduced by a heat shock method (Sambrook, J & Russell, D. W., New York: Cold Spring Harbor Laboratory Press, 2001) to prepare a strain capable of producing 1,4-BDO. The transformed strain was selected and acquired from an LB plate medium including 100 μg/mL of ampicillin and 50 μg/mL of kanamycin.

### Example 2. Verification of t4-BDO Production from Succinate by Culturing the Recombinant Microorganism

In Example 2, it was verified that 1,4-BDO was produced by culturing the recombinant microorganism including 1,4-BDO producing genes which was prepared in Example 1 by using succinate as a major substrate.

Specifically, 1,4-BDO productivity of the microorganism capable of producing 1,4-BDO in which an LDH gene, a ADH gene, and a malate dehydrogenase (MDH) gene were inactivated was compared with respect to whether or not succinate was externally fed and the amount of succinate feeding.

2.1 Verification of 1,4-BDO Production from Succinate by Using Recombinant E. coli W ΔmdhΔldhΔadhE to which pTac15k (sucCD-sucD-4hbd) and pTrc99a (ald-cat2) were Introduced

The transformed strain obtained in Example 1, which was W ΔmdhΔldhΔadh strain to which pTac15k (sucCD-sucD-4hbd) and pTrc99a (ald-cat2) were introduced, was inoculated to 10 mL LB medium including 100 μg/mL of ampicillin and 50 μg/mL of kanamycin and cultured at 30° C. for 12 hours. Then, the culture solution was inoculated to 250 mL flask including 50 mL of MR medium including 15 g/L of glucose, 1 g/L of yeast extract, 100 mM of MOPS, 10 mM of NaHCO₃, 100 μg/mL of ampicillin, and 50 μg/mL of kanamycin and cultured at 30° C. by shaking the flask at 220 rpm of rotation rate. The MR medium included, per 1 L of distilled water, 6.67 g of KH₂PO₄, 4 g of (NH₄)₂HPO₄, 0.8 g of citric acid, 0.8 g of MgSO₄.7H₂O, and 5 mL of a trace metal solution (including 10 g of FeSO₄.7H₂O, 1.35 g of CaCl₂, 2.25 g of ZnSO₄.7H₂O, 0.5 g of MnSO₄.4H₂O, 1 g of CuSO₄.5H₂O, 0.106 g of (NH₄)₆Mo₇O₂₄.4H₂O, 0.23 g of Na₂B₄O₇.10H₂O, and 35% HCl 10 mL per 1 L of distilled water) and the pH of the MR medium was adjusted to 7.0 by using 10 N NaOH. A membrane-type vent cap was installed at the flask so that air might be exchanged. To induce expression of the introduced genes, the microorganism was grown until optical density at 600 nanometers (OD₆₀₀) absorbance reached 0.5 at which time 0.25 mM IPTG was added to the medium. After adding IPTG, culturing was continued for four hours and then sodium succinate was fed all at once at a concentration of 0 g/L, 3 g/L, and 30 g/L, respectively. Afterwards, the culturing was continued for 48 hour under the same conditions. The resulting 1,4-BDO production (mg/L) and succinate consumption (g/L) are shown in Table 1.

1,4-BDO and succinate were analyzed in the following procedure. 1 mL of the culture solution was taken and centrifugated at 13000 rpm for 30 minutes. A supernatant was centrifugated under the same conditions and then 800 μL of the resulting solution was filtered by using a 0.45 μm filter to prepare a sample. 10 μL of the sample was taken and a ultra high performance liquid chromatography instrument (UHPLC, Waters) was used to analyze the quantity of 1,4-BDO in the sample. UHPLC was performed by using Agilent 1100 instrument on which a refractive index detector (RID) was installed. 4 mM H₂SO₄ solution was used as a mobile phase and a BIO-RAD Aminex HPX-87H column was used as stationary phase. The flow rate was 0.7 ml/min. The temperature of both the column and the detector was 50° C.

TABLE 1

| Succinate Fed | 1,4-BDO Production | Succinate Consumption |
|---|---|---|
| (g/L) | (mg/L) | (g/L) |
| 0 | 0 | 0 |
| 3 | 33 | 2.3 |
| 30 | 173 | 6.7 |

As shown in Table 1, according to the culturing method of Example 2, 1,4-BDO was not produced when succinate was not fed, while 1,4-BDO was produced when succinate was fed. In addition, as the amount of the fed succinate was increased, the succinate consumption and the 1,4-BDO production were also increased.

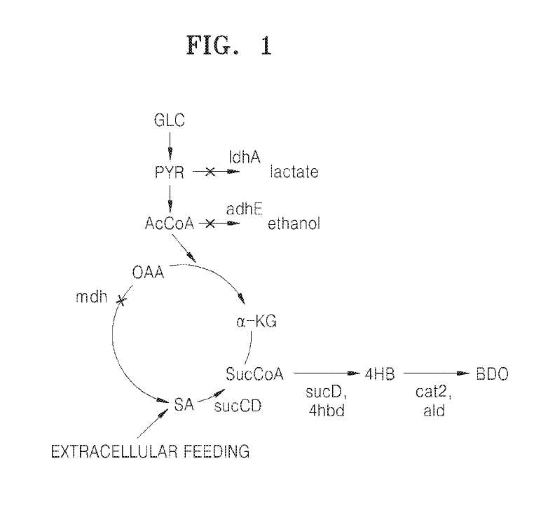

FIG. 1 is a diagram showing the 1,4-BDO production pathway of one embodiment of the microorganism. In FIG. 1, GLC, PYR, AcCoA, OAA, SA, SucCoA, α-KG, 4HB and BDO represents glucose, pyruvate, acetyl-CoA, oxaloaceate, succinic acid, succinyl-CoA, α-ketoglutarate, 4-hydroxybutyrate, and 1,4-butanediol.

As described above, the microorganism according to one aspect of the present disclosure may be capable of producing 1,4-BDO even under aerobic conditions.

According to a method of producing 1,4-BDO according to another aspect of the present disclosure, 1,4-BDO may be efficiently produced.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present disclosure have been described with reference to the figures, it will be understood by those of ordinary skill in the art that various changes in form and details may be made therein without departing from the spirit and scope of the present invention as defined by the following claims.

It should be understood that the exemplary embodiments described herein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-10. (canceled)

11. A method of producing 1,4-BDO comprising culturing in the presence of succinate a genetically engineered microorganism and recovering 1,4-BDO from the culture, wherein the genetically engineered microorganism comprises
a genetic modification that inactivates or attenuates one or more genes encoding a polypeptide converting pyruvate to lactate,
one or more genes encoding alcohol dehydrogenase,
one or more genes encoding a polypeptide converting oxaloacetate to malate;
a genetic modification that increases the expression of one or more genes encoding an enzyme that converts succinate to 4-hydroxybutyrate (4HB) in comparison to a parent microorganism not having the genetic modification; and
a genetic modification that increases the expression of one or more genes encoding an enzyme that converts 4HB to 1,4-butanediol (1,4-BDO) in the genetically engineered microorganism in comparison to a parent microorganism not having the genetic modification.

12. The method in claim 11, wherein additional succinate is fed to the culture during the culturing.

13. The method in claim 11, wherein the culturing is performed at a dissolved oxygen concentration which is from about 1% to about 100% of a saturated concentration.

14. The method in claim 11, wherein the microorganism belongs to Escherichia genus, or Corynebacterium genus.

15. The method in claim 11, wherein the microorganism is E. coli.

16.-19. (canceled)

20. The method in claim 11, wherein the activity of converting succinate to 4-HB is increased by introduction of one or more genes encoding a polypeptide converting succinate to succinyl-CoA, one or more genes encoding a polypeptide converting succinyl-CoA to succinic semialdehyde (SSA), one or more genes encoding a polypeptide converting SSA to 4HB, or a combination thereof in the microorganism.

21. The method in claim 11, wherein the microorganism comprises a genetic modification that increases expression of one or more genes encoding a polypeptide converting 4HB to 4HB-CoA, a polypeptide converting 4HB-CoA to 1,4-BDO, or a combination thereof in the microorganism, wherein the expression is relative to a parent microorganism not having the genetic modification.

22. The method in claim 11, wherein the activity of converting 4HB to 1,4-BDO is increased by introduction of one or more genes encoding a polypeptide converting 4HB to 4HB-CoA, one or more genes encoding a polypeptide converting 4HB-CoA to 1,4-BDO, or a combination thereof in the microorganism.

23. The method in claim 11, wherein the microorganism is an E. coli; wherein the activity of converting succinate to 4-HB is increased by an introduction of one or more genes encoding a polypeptide converting succinate to succinyl-CoA (SSA), one or more genes encoding a polypeptide converting succinyl-CoA to SSA, one or more genes encoding a polypeptide converting SSA to 4HB, or a combination thereof, and the activity of converting 4HB to 1,4-BDO is increased by an introduction of one or more genes encoding a polypeptide converting 4HB to 4HB-CoA, one or more genes encoding a polypeptide converting 4HB-CoA to 1,4-BDO, or a combination thereof.

24. The method in claim 11, wherein the culturing is performed at a dissolved oxygen concentration which is about 10% or more of a saturated concentration.


SEQUENCE LISTING (38 sequences)

SEQ ID NO: 1 - protein, 329 aa, Escherichia coli
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFDQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 2 - DNA, 990 bp, Escherichia coli
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgacgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactaccatctgttgaacgaagccgccttcgatcagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtaattcaggatgacgtattccgtcgcctgtctgcctgccacaacgtgctatttaccgggcaccaggcattcctgacagcagaagctctgaccagtatttctcagactacgctgcaaaacttaagcaatctggaaaaaggcgaaacctgcccgaacgaactggtttaa

SEQ ID NO: 3 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 4 - DNA, 2676 bp, Escherichia coli
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggctcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcgttcgatgaccagtgcaccggcgctaacccgcgttacccgctgatctccgagctgaaacagatcctgctggatacctactacggtcgtgattatgtagaaggtgaaactgcagcgaaaaaagaagccgctccggctaaagctgagaaaaaagcgaaaaaatccgcttaa

SEQ ID NO: 5 - protein, 312 aa, Escherichia coli
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVSKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQSALEGMLDTLKKDIALGEEFVNK

SEQ ID NO: 6 - DNA, 939 bp, Escherichia coli
atgaaagtcgcagtcctcggcgctgctggcggtattggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggtgtagcgcgtaaaccgggtatggatcgttccgacctgtttaacgttaacgccggcatcgtgaaaaacctggtacagcaagtttcgaaaacctgcccgaaagcgtgcattggtattatcactaacccggttaacaccacagttgcgattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgctgtcacaggttcctggcgttagttttaccgagcaggaagtggctgatctgaccaaacgtatccagaacgcaggtactgaagtggttgaagcgaaagccggtggcgggtctgcaaccctgtctatgggccaggcagctgcacgttttggtctgtctctggtacgcgcactgcagggcgaacaaggcgttgtcgaatgtgcctatgttgaaggcgacggtcagtacgcacgtttcttctctcaaccgctgctgctgggtaaaaacggcgtggaagagcgtaaatctatcggtaccctgagcgcatttgaacagagcgcactggaaggtatgctggatacgctgaagaaagatatcgccctgggcgaagagttcgttaataagtaa

SEQ ID NO: 7 - protein, 677 aa, Escherichia coli
MNLHEYQAKQLFARYGLPAPVGYACTTPREAEEAASKIGAGPWVVKCQVHAGGRGKAGGVKVVNSKEDIRAFAENWLGKRLVTYQTDANGQPVNQILVEAATDIAKELYLGAVVDRSSRRVVFMASTEGGVEIEKVAEETPHLIHKVALDPLTGPMPYQGRELAFKLGLEGKLVQQFTKIFMGLATIFLERDLALIEINPLVITKQGDLICLDGKLGADGNALFRQPDLREMRDQSQEDPREAQAAQWELNYVALDGNIGCMVNGAGLAMGTMDIVKLHGGEPANFLDVGGGATKERVTEAFKIILSDDKVKAVLVNIFGGIVRCDLIADGIIGAVAEVGVNVPVVVRLEGNNAELGAKKLADSGLNIIAAKGLTDAAQQVVAAVEGKMSILIDKNTKVICQGFTGSQGTFHSEQAIAYGTKMVGGVTPGKGGTTHLGLPVFNTVREAVAATGATASVIYVPAPFCKDSILEAIDAGIKLIITITEGIPTLDMLTVKVKLDEAGVRMIGPNCPGVITPGECKIGIQPGHIHKPGKVGIVSRSGTLTYEAVKQTTDYGFGQSTCVGIGGDPIPGSNFIDILEMFEKDPQTEAIVMIGEIGGSAEEEAAAYIKEHVTKPVVGYIAGVTAPKGKRMGHAGAIIAGGKGTADEKFAALEAAGVKTVRSLADIGEALKTVLK

SEQ ID NO: 8 - DNA, 2036 bp, Escherichia coli
atgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataatgtccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataa

SEQ ID NO: 9 - protein, 538 aa, Clostridium kluyveri
MSKGIKNSQLKKKNVKASNVAEKIEEKVEKTDKVVEKAAEVTEKRIRNLKLQEKVVTADVAADMIENGMIVAISGFTPSGYPKEVPKALTKKVNALEEEFKVTLYTGSSTGADIDGEWAKAGIIERRIPYQTNSDMRKKINDGSIKYADMHLSHMAQYINYSVIPKVDIAIIEAVAITEEGDIIPSTGIGNTATFVENADKVIVEINEAQPLELEGMADIYTLKNPPRREPIPIVNAGNRIGTTYVTCGSEKICAIVMTNTQDKTRPLTEVSPVSQAISDNLIGFLNKEVEEGKLPKNLLPIQSGVGSVANAVLAGLCESNFKNLSCYTEVIQDSMLKLIKCGKADVVSGTSISPSPEMLPEFIKDINFFREKIVLRPQEISNNPEIARRIGVISINTALEVDIYGNVNSTHVMGSKMMNGIGGSGDFARNAYLTIFTTESIAKKGDISSIVPMVSHVDHTEHDVMVIVTEQGVADLRGLSPREKAVAIIENCVHPDYKDMLMEYFEEACKSSGGNTPHNLEKALSWHTKFIKTGSMK

SEQ ID NO: 10 - DNA, 1617 bp, Clostridium kluyveri
atgagtaaagggattaagaactcgcaactaaaaaaaaaaaatgtgaaggccagtaatgtggcagaaaagattgaagagaaagttgaaaaaacggataaggttgttgaaaaagccgctgaggttacagagaaacggattagaaacctgaagctgcaggagaaagttgttacagcggatgtggcggctgatatgattgaaaatggcatgattgtggcaatcagcggttttactccgtccggttatccaaaggaagtccctaaagcactgactaaaaaagttaatgccctggaggaggagttcaaggtcaccttatataccgggtcaagcacgggggccgacatcgacggggaatgggcaaaggcaggaatcatagaacggcgtatcccctaccagacaaattctgacatgcgaaaaaaaataaatgacggttctattaagtacgctgatatgcatttaagccatatggctcaatatattaattattctgtcattcctaaagtcgatatagctataatagaagcggtagctattacggaagaaggggatataattccttcgacgggaattggcaataccgcgacttttgtggaaaacgcggacaaagtgatagtggaaattaacgaagcccaaccgctggaattggagggcatggcagacatatacacattaaaaaaccccccgcgtagagagccgattccaatagttaatgctggcaatcgcatagggaccacatatgtgacctgtggctcggaaaaaatctgcgccatcgtcatgacaaatacgcaagacaaaacaagacctcttacagaggtgtctcctgtatctcaggccatctccgacaatctgataggttttttaaacaaagaagtggaagagggcaaattacctaaaaacctgctccccatacagtcaggagttggtagtgtcgcaaatgcggttttggccggtctttgtgaatcaaactttaaaaacctaagttgttacacggaggttatccaggatagcatgctgaagcttataaaatgtggaaaagcagatgtggtgtcaggcacctccataagtccatcaccggagatgctgcctgagttcatcaaggacataaacttctttagagaaaagatagtattaagaccacaggaaatcagcaataacccagagatagcacgcagaatcggtgtgatatccataaacaccgccttggaagtagacatatatggtaatgtaaacagtacgcacgttatgggaagcaaaatgatgaatggcataggcggttctggcgactttgcccgcaatgcatatctcactatcttcactacagagtctatcgccaaaaaaggcgatatctcaagcatagtgcctatggtatcccatgtggatcataccgaacatgatgtaatggtcatcgttaccgaacagggagtagcggatctgcgcggtctttctcctagggaaaaggcggtggctataatcgaaaattgcgttcatccggactataaggatatgctgatggagtattttgaagaagcgtgcaaatcgtcaggtgggaacaccccacacaatcttgaaaaagctctttcatggcacacaaaatttataaaaacgggtagcatgaaataa

SEQ ID NO: 11 - protein, 451 aa, Porphyromonas gingivalis
MEIKEMVSLARKAQKEYQATHNQEAVDNICRAAAKVIYENAAILAREAVDETGMGVYEHKVAKNQGKSKGVWYNLHNKKSIGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 12 - DNA, 1356 bp, Porphyromonas gingivalis
atggaaataaaagagatggtgtcgttggcaaggaaagctcagaaggaatatcaagcgacccataatcaagaagcagttgataacatttgccgagctgcagcaaaagtgatttatgaaaatgcagctatactggctcgcgaagcagtagacgaaaccggcatgggcgtatatgaacataaagtggccaagaatcaggggaaatccaaaggcgtctggtacaatttgcacaataaaaaatcgatcggtatcttaaatatagacgagagaaccgggatgatcgagatagcaaaacctatcggggttgttggagccgtaaccccgacgacaaacccgattgtgactccaatgagcaacatcatttttgcccttaagacatgcaatgccattattatcgccccacatcccagatccaaaaaatgctcagcacatgcagttcgtctgataaaggaagcaatcgctccgtttaatgtcccggagggaatggttcagatcattgaagagcccagcatcgagaaaactcaggaactaatgggcgccgtggatgtggtagttgcgacgggtggtatgggtatggtgaaatctgcatattcttcagggaagccttcttttggtgtaggagccggtaacgttcaagtgatcgtggatagtaatatcgattttgaagctgcggcagaaaaaattatcaccggccgtgctttcgacaatgggatcatctgttcaggcgaacagagtatcatctacaacgaagctgacaaggaagctgtcttcacagccttccgcaaccatggtgcatatttttgtgatgaagcggagggagatcgggcccgtgctgcgatttttgagaatggcgccatcgcgaaagatgtagtcggccagagcgttgcctttatcgcgaagaaagcaaatatcaatataccggagggtacccgtattctggttgttgaagctcgcggcgtcggagcagaggatgtcatatgtaaggaaaaaatgtgtccagttatgtgcgccttaagctacaagcacttcgaggaaggtgtagaaatcgcacgtacgaacttggccaacgaaggtaacggccatacctgtgcgatccattccaacaatcaggcgcatatcatactggcaggttcagaactgacggtttcgcggatcgtggtcaatgcgccgagtgccactacagcaggcggtcacatccaaaatggtctggcagtgacaaatacgctcggatgcgggagttggggtaataactctatctccgagaactttacttataaacacctgttaaacattagccgcatagcgccgcttaattcaagcattcacattcctgatgacaaagagatctgggaactctaa

SEQ ID NO: 13 - protein, 371 aa, Porphyromonas gingivalis
MQLFKLKSVTHHFDTFAEFAKEFCLGERDLVITNEFIYEPYMKACQLPCHFVMQEKYGQGEPSDEMMNNILADIRNIQFDRVIGIGGGTVIDISKLFVLKGLNDVLDAFDRKIPLIKEKELIIVPTTCGTGSEVTNISIAEIKSRHTKMGLADDAIVADHAIIIPELLKSLPFHFYACSAIDALIHAIESYVSPKASPYSRLFSEAAWDIILEVFKKIAEHGPEYRFEKLGEMIMASNYAGIAFGNAGVGAVHALSYPLGGNYHVPHGEANYQFFTEVFKVYQKKNPFGYIVELNWKLSKILNCQPEYVYPKLDELLGCLLTKKPLHEYGMKDEEVRGFAESVLKTQQRLLANNYVELTVDEIEGIYRRLY

SEQ ID NO: 14 - DNA, 1116 bp, Porphyromonas gingivalis
atgcaactgttcaaactgaaatcagtcacacatcacttcgatactttcgcggaatttgccaaagagttctgtcttggagaacgtgatttagtaattaccaacgaattcatttacgaaccgtatatgaaggcatgtcagttgccctgccattttgttatgcaggagaaatatgggcaaggcgagccatctgacgagatgatgaataacatcttggcagacatccgtaatatccagtttgaccgcgtgatcggtattgggggtggtacggttattgacatctcgaaattatttgtgctgaaaggactaaatgatgtgctcgatgcgttcgatcgcaagataccgctgattaaagagaaagaactgatcattgtgcccaccacatgcgggacgggtagcgaggtgacgaatatttcgatcgcggagatcaaaagccgtcataccaaaatgggtttggctgacgatgctattgttgcagaccacgcgatcatcataccagagcttctgaaaagcctgccgttccatttttatgcatgcagtgcaatagatgctctgatccatgccatcgagtcatatgtttctcctaaagccagtccatattctcgtctgttcagtgaggcggcatgggatattatcctggaggtattcaagaaaatagccgaacacggccctgaataccgctttgagaagctgggagaaatgatcatggcctccaactatgctggtatagccttcgggaatgcaggcgtgggtgccgttcacgctctaagctatccattgggaggcaattatcatgtgccgcatggcgaggctaactatcagttttttacagaggtctttaaagtataccaaaagaaaaatcctttcggctatatagtcgaactcaactggaagctgtccaagattctgaactgtcagcctgaatacgtctatccgaaactggatgagttactcggctgtcttctgaccaaaaaaccgctgcacgaatacggcatgaaagatgaagaggtacgtggatttgcggaatcagtgcttaagactcagcagcggttgctcgcgaataattatgttgagcttactgttgatgaaattgaaggtatctacagacgactgtactaa

SEQ ID NO: 15 - protein, 431 aa, Porphyromonas gingivalis
MKDVLAEYASRIVSAEEAVKHIKNGERVALSHAAGVPQSCVDALVQQADLFQNVEIYHMLCLGEGKYMAPEMAPHFRHITNFVGGNSRKAVEENRADFIPVFFYEVPSMIRKDILHIDVAIVQLSMPDENGYCSFGVSCDYSKPAAESAHLVIGEINRQMPYVHGDNLIHISKLDYIVMADYPIYSLAKPKIGEVEEAIGRNCAELIEDGATLQLGIGAIPDAALLFLKDKKDLGIHTEMFSDGVVELVRSGVITGKKKTLHPGKMVATFLMGSEDVYHFIDKNPDVELYPVDYVNDPRVIAQNDNMVSINSCIEIDLMGQVVSECIGSKQFSGTGGQVDYVRGAAWSKNGKSIMAIPSTAKNGTASRIVPIIAEGAAVTTLRNEVDYVVTEYGIAQLKGKSLRQRAEALIAIAHPDFREELTKHLRKRFG

SEQ ID NO: 16 - DNA, 1296 bp, Porphyromonas gingivalis
atgaaagacgtgttagcggaatatgcctcccgaattgtttcggccgaagaggcagtcaaacatatcaaaaatggagagcgtgtcgctttatcacatgctgccggagttcctcagagttgtgttgacgcactggtgcaacaggcggacctgtttcagaatgtggagatttaccacatgctgtgtctcggcgaaggaaaatatatggcacctgaaatggcccctcacttccggcacataaccaattttgttggtggtaactctcgtaaagcagtggaggaaaatagagccgacttcattccggtattcttttatgaagtgccatcaatgattcggaaagatatccttcatatagatgtggccattgtccaactctcaatgccagatgagaatggttactgcagctttggcgtatcttgcgattatagcaaaccggcggcggaatcggcgcatttagttattggggaaatcaaccgtcagatgccatatgtgcatggtgacaacttgattcacatatcgaagttggattacatcgtgatggcggattacccaatttattctctggcgaagcccaaaatcggagaagtagaggaagctatcggccgtaactgtgccgagcttattgaagatggtgccaccctacagctgggtatcggcgcgattccggatgcagctctgctgtttctgaaggacaaaaaagatctggggattcatactgaaatgttctccgatggcgttgttgaactggtgcgcagtggtgtaattactggaaaaaaaaagacattgcatcccggtaagatggtcgcgacgtttcttatgggatcagaagacgtgtatcatttcatcgacaagaatccggatgtggaactgtatccggttgattacgtcaatgatccgagggttatcgctcagaatgataatatggtcagcatcaatagctgtatcgagatcgatctaatgggccaagtggtgagcgagtgcataggctccaaacagtttagtggcaccgggggtcaagtagattatgtccgcggggcagcttggtctaaaaacggcaaaagcatcatggcaattccctcaacagccaaaaacggtactgcatctcggatagttcctataattgcagagggcgctgctgtaacaaccctccgcaacgaagtcgactacgttgttacggaatatgggatagcacagttaaaaggtaagagtttgcgtcagcgcgcagaagctcttattgcgatagcccacccggactttagagaggaactgacgaagcatctgcgcaaacgttttggttaa

SEQ ID NO: 17 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 18 - DNA, 2577 bp, Clostridium acetobutylicum
atgaaagtcaccaaccagaaagagctgaaacagaaactgaacgaactgcgtgaagcccagaagaagttcgctacgtacacccaggaacaggtggacaaaatcttcaaacagtgcgcgattgctgctgcaaaagaacgtatcaacctggctaaactggccgtggaagagacgggcattggtctggtggaagacaagatcatcaaaaaccactttgcggcggaatacatctacaacaagtacaaaaacgagaaaacttgcggcatcatcgaccacgatgattccctgggcatcaccaaagtggcagaacctatcggtattgttgcagcaattgtaccgactactaacccgacttctaccgctattttcaagtctctgatttctctgaaaacccgcaacgcgattttcttctctccgcacccacgtgcgaaaaaatccaccatcgctgccgccaaactgatcctggacgcggcagttaaagcgggtgctccgaaaaatatcattggttggatcgatgaaccgtctatcgaactgagccaggatctgatgtccgaagcagacattatcctggcaaccggcggtccgtctatggtaaaagccgcctactcttctggcaaaccggcaattggtgttggtgctggtaacacgccggcgattatcgacgagtccgcagacatcgatatggcagtttcctctatcattctgtccaaaacctacgataacggcgtgatctgcgcgagcgaacagtccatcctggttatgaattctatctatgaaaaggtcaaggaagaatttgttaagcgtggcagctacatcctgaaccagaacgagatcgcgaaaatcaaagaaactatgttcaaaaacggtgccatcaatgccgacatcgtcggcaaatctgcttacattattgccaaaatggctggtatcgaagtgccgcagaccacgaagatcctgatcggtgaggtacagagcgttgaaaagtctgaactgttctctcatgaaaaactgtccccggtcctggctatgtacaaagtaaaagacttcgacgaagcactgaaaaaagcgcaacgtctgatcgagctgggtggtagcggccacacctctagcctgtacatcgacagccagaacaacaaagataaagttaaagaattcggcctggcaatgaaaaccagccgcacctttattaacatgccttctagccaaggtgcttctggcgacctgtataacttcgctattgcgccttcctttaccctgggttgcggtacctggggcggtaacagcgtttcccaaaacgttgaaccgaaacacctgctgaacattaaatctgtagcagaacgccgtgagaacatgctgtggtttaaagttccgcagaaaatctacttcaagtacggttgtctgcgcttcgctctgaaagaactgaaggatatgaacaagaaacgtgcgttcatcgtgactgataaagatctgttcaaactgggctacgttaacaaaatcactaaagtactggacgaaatcgatattaagtattccatctttaccgacatcaaatctgacccgaccatcgattccgtaaaaaagggtgctaaggaaatgctgaacttcgaaccggacactattatcagcatcggcggtggctctccgatggatgcagcaaaagtgatgcatctgctgtacgaatacccggaagcggaaatcgaaaacctggcgatcaatttcatggacatccgtaaacgtatctgcaattttccgaagctgggtacgaaagccatttccgttgcgattccgactaccgcgggtactggttctgaagcgaccccgttcgctgttattactaacgatgaaactggtatgaaatacccactgacgagctatgagctgaccccaaacatggcaatcattgataccgagctgatgctgaatatgccgcgtaaactgaccgcggcgactggcatcgacgccctggttcacgcgatcgaagcttatgtttctgtcatggccaccgattatacggacgaactggctctgcgtgctatcaaaatgattttcaaatatctgcctcgcgcgtacaagaacggcaccaacgatattgaggctcgtgaaaaaatggcacacgccagcaacatcgcaggcatggcattcgctaacgcttttctgggcgtatgccattccatggctcataaactgggtgcaatgcaccacgttccacacggcatcgcgtgtgcggtgctgatcgaagaggtgatcaaatacaacgctactgactgtccgactaaacaaaccgcgtttccgcagtacaaatccccaaatgcgaaacgtaaatatgcggagatcgccgaatatctgaacctgaaaggcacctccgacaccgaaaaagtgaccgctctgattgaagccatcagcaaactgaaaattgacctgtctatcccgcagaacatcagcgcggcaggtatcaacaaaaaagatttctataacaccctggataaaatgagcgagctggcgttcgatgaccagtgtacgaccgcaaacccgcgctacccgctgatctccgaactgaaagacatttatattaaatccttctaa

SEQ ID NO: 19 - protein, 468 aa, Clostridium beijerinckii
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 20 - DNA, 1407 bp, Clostridium beijerinckii
atgaataaggatacgttgatcccgaccaccaaggatctgaaggtcaagaccaatggcgagaacatcaatcttaaaaactacaaagacaacagctcctgtttcggagtgtttgaaaatgtggaaaacgcgatctcctcagcagtacacgcgcaaaagatcctctctttgcactacactaaggaacagcgtgaaaagattatcacggagatccgcaaagcggcactgcagaacaaagaggtcctggctacaatgatcttggaggagacacatatgggtcgctacgaggacaagatcctcaagcacgagcttgttgctaagtacacccccggcaccgaggatcttaccaccaccgcctggtctggcgataatggactgaccgttgtggaaatgtccccctacggcgttatcggggcaattaccccaagcacaaacccaaccgaaaccgtgatttgtaactcgatcggaatgatcgccgcaggtaacgctgtggttttcaacggccacccatgcgcaaagaagtgcgttgcatttgccgtggagatgatcaacaaggcaattatctcatgcggtggtcctgaaaacctcgtcactaccattaagaatccaactatggagtcgcttgatgctatcattaagcacccatcgatcaagcttctctgtggaactggcggccctggcatggtcaaaacgctcctgaacagcgggaaaaaggcgattggagccggtgcaggtaatccgcccgtcatcgtggacgatacggcagatattgagaaggccggtcgttccatcatcgaaggctgctcatttgataacaacctgccgtgcattgctgagaaagaagttttcgttttcgagaacgttgccgatgaccttatttccaatatgttgaagaataatgcagtgatcatcaacgaagaccaagtttccaaactgatcgatctcgtccttcagaaaaacaacgagactcaggaatatttcattaacaagaagtgggtgggcaaagacgcaaagctgttcttggatgagattgacgtggagagcccttccaacgtcaagtgcattatctgtgaagtcaacgctaaccatcctttcgtgatgacggaattgatgatgccaatcctgccgattgttcgagtaaaagacattgacgaagctatcaagtacgcgaaaatcgccgaacagaaccgcaagcactctgcttatatctactctaagaacattgacaatctgaaccggtttgaacgggagatcgacactaccatctttgtcaaaaacgcgaaatccttcgctggcgtgggctatgaagctgagggattcaccaccttcaccattgcggggagcaccggtgaaggcatcacttctgcccgcaacttcacccgccagcgccgttgcgtactcgccggttaa

SEQ ID NO: 21 - DNA, 71 bp, Artificial Sequence
  Synthetic (ldhA KO primer_up)
atgaaactcgccgtttatagcacaaaacagtacgacaagaagtacctgcataggtgacactatagaacgcg

SEQ ID NO: 22 - DNA, 70 bp, Artificial Sequence
  Synthetic (ldhA KO primer_do)
ttaaaccagttcgttcgggcaggtttcgcctttttccagattgcttaagttagtggatctgatgggtacc

SEQ ID NO: 23 - DNA, 20 bp, Artificial Sequence
  Synthetic (primer_up)
tacactaagcatagttgttg

SEQ ID NO: 24 - DNA, 20 bp, Artificial Sequence
  Synthetic (primer_do)
ctttcttcattgtggttctc

SEQ ID NO: 25 - DNA, 71 bp, Artificial Sequence
  Synthetic (adhE KO primer)
atggctgttactaatgtcgctgaacttaacgcactcgtagagcgtgtaaataggtgacactatagaacgcg

SEQ ID NO: 26 - DNA, 70 bp, Artificial Sequence
  Synthetic (adhE KO primer)
ttaagcggattttttcgcttttttctcagctttagccggagcggcttctttagtggatctgatgggtacc

SEQ ID NO: 27 - DNA, 20 bp, Artificial Sequence
  Synthetic (primer-up)
caccgcactgactatactct

SEQ ID NO: 28 - DNA, 20 bp, Artificial Sequence
  Synthetic (primer-do)
gatgaaggctaatgctgtcg

SEQ ID NO: 29 - DNA, 71 bp, Artificial Sequence
  Synthetic (mdh KO primer_up)
atgaaagtcgcagtcctcggcgctgctggcggtattggccaggcgcttgctaggtgacactatagaacgcg

SEQ ID NO: 30 - DNA, 70 bp, Artificial Sequence
  Synthetic (mdh KO primer_do)
ttacttattaacgaactcttcgcccagggcgatatctttcttcagcgtattagtggatctgatgggtacc

SEQ ID NO: 31 - DNA, 20 bp, Artificial Sequence
  Synthetic (primer_up)
ggttcctgattacggcaatt

SEQ ID NO: 32 - DNA, 20 bp, Artificial Sequence
  Synthetic (primer_do)
attcaggaatatccggcaac

SEQ ID NO: 33 - DNA, 32 bp, Artificial Sequence
  Synthetic (primer)
gctagaattcatgaacttacatgaatatcagg

SEQ ID NO: 34 - DNA, 31 bp, Artificial Sequence
  Synthetic (primer)
gcaaggtaccttatttcagaacagttttcag

SEQ ID NO: 35 - DNA, 58 bp, Artificial Sequence
  Synthetic (primer)
gaaaactgttctgaaataagtcacacaggaaacagaattcatggaaataaaagagatg

SEQ ID NO: 36 - DNA, 58 bp, Artificial Sequence
  Synthetic (primer)
ttcagtttgaacagttgcatgaattctgtttcctgtgtgattagagttcccagatctc

SEQ ID NO: 37 - DNA, 58 bp, Artificial Sequence
  Synthetic (primer)
aagagatctgggaactctaatcacacaggaaacagaattcatgcaactgttcaaactg

SEQ ID NO: 38 - DNA, 47 bp, Artificial Sequence
  Synthetic (primer)
cgactctagaggatccccggttagtacagtcgtctgtagataccttc